(19) United States
(12) Patent Application Publication
(10) Pub. No.: US 2023/0081282 A1
(43) Pub. Date: Mar. 16, 2023

(54) SYSTEMS AND METHODS FOR DESIGNING SELF-ASSEMBLED NANOSTRUCTURES

(71) Applicant: Arizona Board of Regents on Behalf of Arizona State University, Tempe, AZ (US)

(72) Inventors: Petr Sulc, Scottsdale, AR (US); Flavio Romano, Venezia (IT); John Russo, Rome (IT); Lukas Kroc, Praha (CZ)

(21) Appl. No.: 17/192,305

(22) Filed: Mar. 4, 2021

Related U.S. Application Data
(60) Provisional application No. 62/985,204, filed on Mar. 4, 2020.

(51) Int. Cl. G06F 30/10

(52) U.S. Cl. CPC G06F 30/10

(57) ABSTRACT

A general framework which transforms the inverse problem of self-assembly of colloidal crystals into a Boolean satisfiability problem for which solutions can be found numerically is described herein. Given a reference structure and the desired number of components, our approach produces designs for which the target structure is an energy minimum, and also allows to exclude solutions that correspond to competing structures.

### CROSS REFERENCE TO RELATED APPLICATIONS

The present document is a Non-Provisional patent application that claims benefit to U.S. Provisional Patent Application Ser. No. 62/985,204 filed 4 Mar. 2020 which is herein incorporated by reference in its entirety.

### GOVERNMENT SUPPORT

This invention was made with government support under N00014-20-1-2094 awarded by the Office of Naval Research. The government has certain rights in the invention.

### FIELD

The present disclosure generally relates to particle self-assembly, and in particular, to a system and associated method for designing patchy interactions to self-assemble arbitrary structures.

### BACKGROUND

Self-assembly is a broad category of processes by which elementary components organize themselves into ordered structures. Inspired by the ubiquity of self-assembly in the biological world, nanotechnology has long looked at self-assembly as the most promising avenue for the bottom-up realization of structures ranging from nanometer to micrometer scale with specific functional properties.

Successful experimental examples of self-assembly processes include the realization of two-dimensional lattice, fully three-dimensional crystals, and polyhedral shells. On the molecular scale, perhaps the most successful results were obtained using DNA nanotechnology. Short strands of DNA are prepared with sequences that can form the maximum number of base pairs only by arranging into the desired target structure. This principle has been exploited to self-assemble DNA origami, where thousands of short molecules can be designed to take almost any two- or three-dimensional shape. Very recently, DNA origami have been crystallized into three dimensional superlattices. At the colloidal scale, promising strategies for self-assembly include DNA-functionalized particles and patchy particles. In the former case, a mixture is obtained from colloids whose surface is randomly decorated with single strands of DNA such that particles of different types can selectively bind to each other. This strategy has led to the self-assembly of the double diamond (or B32) crystal. In the case of patchy particles, colloidal particles acquire anisotropic interactions either via their shape or via chemical patterning of their surface. Hybrid solutions where patchy interactions are realized by attaching DNA sequences at well-defined positions have also been proposed.

The experimental methodologies so far described, while very successful, are system-specific and hard to generalize. In many cases, a theoretical understanding of why certain structures have self-assembled from elementary building blocks is lacking. The search for the general principles behind the inverse self-assembly problem has attracted several theoretical investigations. Instead of predicting which structures self-assemble out of specific building blocks, the inverse problem is concerned with designing building blocks that form a specific target. So far, two types of approaches have emerged: optimization algorithms and geometrical strategies. In optimization algorithms the pair potential is tuned to minimize the energy of a target structure. While powerful and general, the major limitation of this approach is that the level of control over the shape of the pair-potential is in most cases far beyond current experimental possibilities. The geometric approach to self-assembly instead uses specific interactions to match the geometrical properties of the target structure to kinetically guide the assembly process. The following interaction properties are usually tuned to match the target structure: shape, directionality, selective binding, and torsional interactions between neighbors. Geometrical approaches allow experimentally realizable systems to self-assemble into specific structures, but the process of de-signing the potential is system-specific and requires ad-hoc solutions. A clear example of these limitations is the self-assembly of the colloidal diamond structure, which usually requires either torsional interactions or hierarchical assembly to avoid the formation of stacking faults. Importantly, some of these features lack a convincing experimental counterpart.

It is with these observations in mind, among others, that various aspects of the present disclosure were conceived and developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a diagram showing a schematic representation of a unit cell of a tetrastack lattice (target configuration) including 16 positions, each bound to its neighbors by a plurality of numbered slots;

FIG. 1B is a diagram showing a topology representative of the unit lattice for the unit cell of FIG. 1A, showing each lattice position connected to 6 other positions;

FIG. 1C is a diagram showing a patchy particle (PP) with 6 patches, each patch including a different patch type that can be positioned into the lattice for the unit cell of FIG. 1A such that each patch fills a slot of the unit cell;

FIG. 1D is a diagram showing an interaction matrix showing which patch types of the PP of FIG. 1C interact;

FIG. 1E is a diagram showing a solution found by a Boolean satisfiability solver, the solution including assigned PP species and orientation such that each lattice position of the unit cell of FIG. 1A is filled and all conditional clauses imposing arrangement restrictions are satisfied;

FIG. 2A is a diagram showing a simulated tetrastack (TS) assembled lattice structure and a corresponding graphical representation showing energy per particle over the course of simulations at differing temperatures;

FIG. 2B is a diagram showing a simulated diamond cubic (DC) assembled lattice structure and a corresponding graphical representation showing energy per particle over the course of simulations at differing temperatures;

FIG. 2C is a diagram showing a simulated clathrate Si34 (CSi34) assembled lattice structure and a corresponding graphical representation showing energy per particle over the course of simulations at differing temperatures;

FIG. 3A is a diagram showing further simulations of diamond cubic (DC) assembled lattice structure and a corresponding graphical representation showing energy as a function of Monte Carlo sweeps;

FIG. 3B is a diagram showing further simulations of clathrate Si34 (CSi34) assembled lattice structure and a corresponding graphical representation showing energy as a function of Monte Carlo sweeps;

FIG. 4 is a diagram showing a general framework for determining a patch type scheme for at least one particle species of patchy particles such that the patchy particles assemble into a target structure;

FIG. 5 is a diagram showing an overall methodology for determining a patch type scheme for at least one particle species of patchy particles such that the patchy particles assemble into a target structure; and

FIG. 6 is a simplified diagram showing an exemplary computing system for implementation of the system of FIGS. 1A-5.

Corresponding reference characters indicate corresponding elements among the view of the drawings. The headings used in the figures do not limit the scope of the claims.

### DETAILED DESCRIPTION

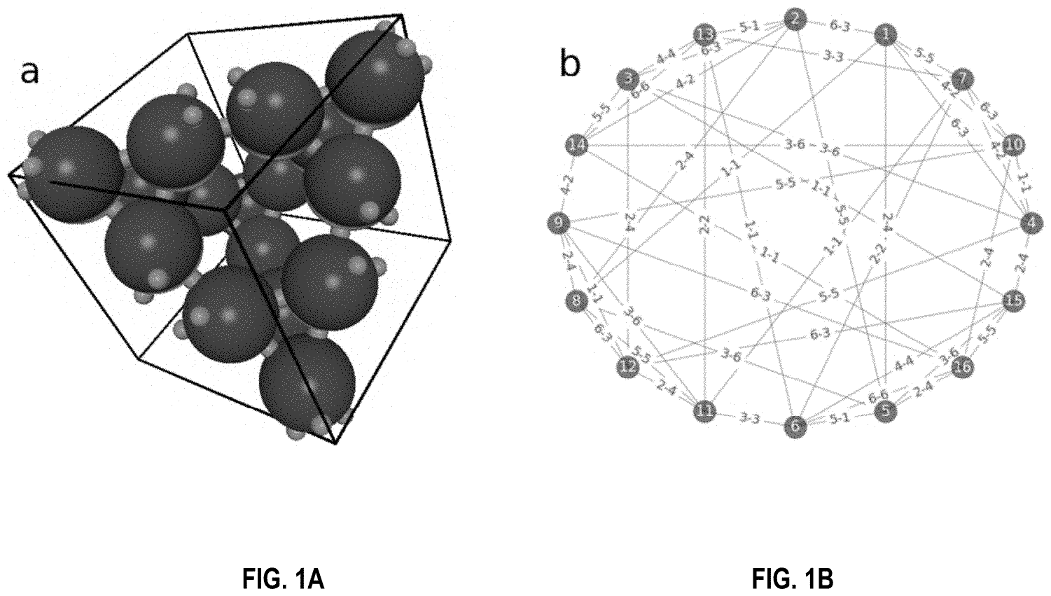

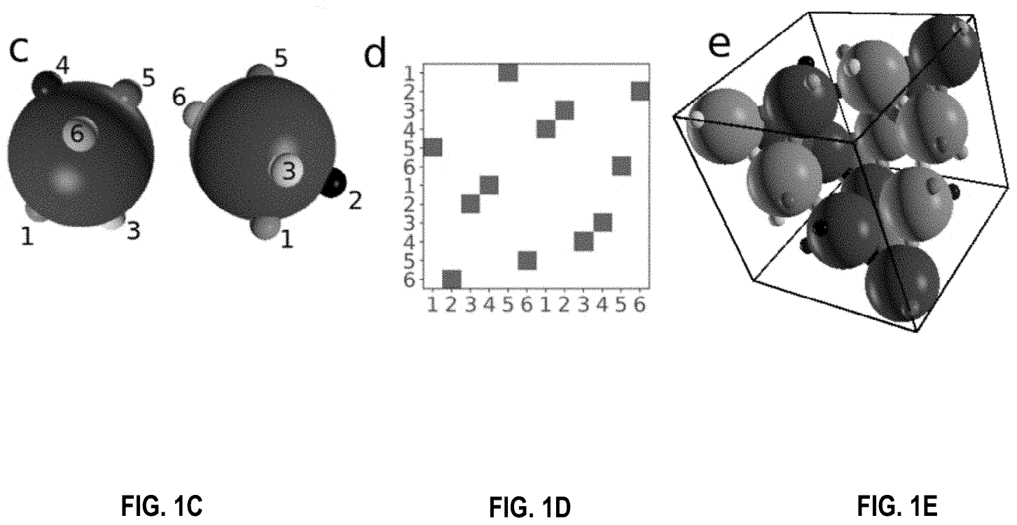

A general framework for designing self-assembling systems of patchy particles (PP) into any arbitrary structure is disclosed herein, with the option to exclude the formation of competing structures that are identified in simulations. The framework described herein focuses on PP systems that have geometric properties, such as the number and placement of patches on a particle that reflect the local environment of the target lattice. To introduce selectivity in the framework, each patch is assigned a patch type, or “type”, that encodes its binding properties and N_{c} is defined as a number of different patch types on a given particle. Binding is allowed only between patches that have compatible types as specified by an interaction matrix. In some embodiments, the framework does not impose any torsional restrictions and all bonds have the same strength. While all particles have the same placement of the patches, the framework allows for the possibility of having Nₛ different PP “species”, which are defined by an arrangement of the typing of their patches. Relative concentrations of each type of PP species are also free parameters. This system can be realized experimentally with patches based on single-stranded DNA thanks to the selective binding of DNA sequences. To simplify sequence design, the framework imposes that each patch is assigned a type that can only bind with one other type (which can be the same in the case of self-complementarity). The goal is to determine the patch typing for each PP species and type interaction matrix so that the PPs assemble into a desired structure. Referring to the drawings, embodiments of a system and associated method for designing patchy interactions to self-assemble arbitrary structures are illustrated and generally indicated as 100 in FIGS. 1-6.

The target structure is described by a unit cell, shown in FIG. 1A, including l∈[1,L] particles. The unit cell can be a combination of two or more true unit cells of the target lattice. The positions of the patches in the target lattice (slots) are labeled as k∈[1,Nₚ], from which a list of neighboring slots is computed, as illustrated in FIG. 1B. Designed PPs can be of different species s∈[1,Nₛ], and have p∈[1,Nₚ] patches on their surface which can take a patch type c∈[1,N_{c}], as shown in FIG. 1C. o represents one of the Nₒ possible orientations of each particle, and it is uniquely identified by a map between its patches and the patch slots they occupy. Not all mappings are possible, only those that can be reached by a physical rotation of the particle. FIG. 1D illustrates an interaction matrix that shows interaction compatibility between patch types of different PP species. FIG. 1E illustrates a solution formed by two PP species that forms the unit cell lattice structure of FIG. 1A by selected arrangement and orientation of each modeled PP according to their patch types described in the interaction matrix of FIG. 1D.

A brute-force search of all possible combinations of (i) patch type arrangements for all particle species

$$( \begin{matrix}{N}_{c} {N}_{s} \\ {N}_{p}\end{matrix} ) ,$$

(ii) rotations and symmetry operations of each particle, up to Nₚ^{L}! and (iii) interaction matrix between patch types,

$$( \begin{matrix}( {N}_{c} + 1 ) / 2 \\ {N}_{c}\end{matrix} ) ,$$

becomes intractable with increasing N_{c}, Nₛ, Nₚ, and L even if they are relatively small. Instead, and this is a crucial contribution of this disclosure, the problem is mapped to a Boolean satisfiability problem (SAT) where recent algorithmic developments have dramatically advanced the present ability to solve problems involving tens of thousands of variables and millions of constraints. In some embodiments, the framework uses a publicly available SAT solver that can find solutions to the design problems considered here in time ranging from few seconds up to one hour.

Mapping the particle design onto a SAT problem requires the definition of (i) binary variables xᵢ that describe the PPs' patch typing for each particle species and the type interaction matrix and (ii) binary clauses Cⱼ that represent the constraints that the variables need to satisfy, such as the ability to form all the bonds in the target lattice. Each binary clause contains a subset of the variables xᵢ (or their negation ¬xᵢ) connected by an OR statement, and a solution is found whenever a combination of values of the xᵢ satisfies all clauses at the same time. Formally, this corresponds to finding a set of variables xi such that C₁ΛC₂ΛC₃Λ . . . is true. The SAT mapping can also be used to prove the impossibility of achieving some (desired or unwanted) binding pattern with a given combination of input parameters Nₛ and N_{c} by proving the absence of solutions to the associated SAT problem. This is crucial since it allows filtering of undesired binding patterns, which may represent competing global arrangements (i.e., another crystal form) or local arrangements (kinetic traps).

A design problem thus translates into a set of binary variables and clauses, as defined in Table I. In order, the clauses enforce that (i) C^{int}: each type is compatible with exactly one type, (ii) C^{pcol}: each patch is assigned exactly one type, (iii) C^{L}: each lattice position is occupied by a single PP species with one assigned orientation, (iv) C^{lint}: types of patches that interact in the target lattice can bind to each other according to the interaction matrix, (v) C^{LS}: the slots in each lattice position are set to have the type of the patch occupying them, Cₛ^{alls.}: all Nₛ particle species are used for the lattice assembly, (vii) C^{all c.}: all N_{c} patch types are used in the solution. The final SAT problem is a conjunction of all clauses (i)-(vii). The conditions (vi) and (vii) are used to avoid getting trivial solutions such as having a single PP species with all patches typed by the same self-complementary type. It allows formulation of the SAT problems for different combinations of Nₛ and N_{c} to see for which the solutions exist.

TABLE 1
SAT Clauses and Variables

| Id | Clauses | Boolean expression |
|---|---|---|
| (i) | C_{c}_{ᵢ}_{, c}_{ⱼ}_{, c}_{ₖ}^{int} | ¬x_{c}_{ᵢ}_{, c}_{ⱼ}^{int} ∨ ¬x_{c}_{ᵢ}_{, c}_{ₖ}^{int} |
| (ii) | C_{s, p, c}_{ₖ}_{, c}_{ₗ}^{pcol} | ¬x_{s, p, c}_{ₖ}^{pcol} ∨ ¬x_{s, p, c}_{ₗ}^{pcol} |
| (iii) | C_{l, s}_{ᵢ}_{, o}_{ᵢ}_{, s}_{ⱼ}_{, o}_{ⱼ}^{L} | ¬x_{l,} ₛ_{ᵢ}_{,} ₒ_{ᵢ}^{L} ∨ ¬x_{l,} ₛ_{ⱼ}_{,} ₒ_{ⱼ}^{L} |
| (iv) | Cₗ_{ᵢ}_{, k}_{ᵢ}_{, l}_{ⱼ}_{, k}_{ⱼ}_{, c}_{ᵢ}_{, c}_{ⱼ}^{lint} | (xₗ_{ᵢ}_{, k}_{ᵢ}_{, c}_{ᵢ}^{A} ∧ xₗ_{ⱼ}_{, k}_{ⱼ}_{, c}_{ⱼ}^{A}^{) ⇒ x}_{c}_{ᵢ}_{, c}_{ⱼ}^{int} |
| (v) | C_{l, s, o, c, k}^{LS} | x_{l, s, o}^{L} ⇒ (x_{l, k, c}^{A} ⇔ x_{s, ϕ}_{ₒ}_{(k), c}^{pcol}) |
| (vi) | Cₛ^{all s.} | ∨_{∀l, o} x_{l, s, o}^{L} |
| (vii) | C_{c}^{all c.} | ∨_{∀s, p} x_{s, p, c}^{pcol} |

For the lattice design problems considered in the present disclosure, the number of binary variables ranges from about 10³ to 10⁵, and the number of clauses ranges from approx. 10⁴ to 10⁷, which were found to be within reach of a commonly used SAT solver. If a solution is found in terms of the binary variables x, it can be straightforwardly converted into human-readable form by listing the variables x_{s,p,c}^{pcol} and x_{c}_{ᵢ}_{,c}_{ⱼ}^{int} that are 1, as their subscripts will specify respectively i) the type c of patch p in PP species s, ii) the compatible types cᵢ and cⱼ. Additionally, the present framework allows the user to quickly check if a specific combination of PP species with a patch typing and type interaction matrix can satisfy a given lattice geometry. The framework uses clauses (i)-(iv) discussed above, and additionally add clauses that constrain the variables x^{pcol} and x^{int} accordingly for the set of PPs that need to be checked. If such a SAT problem is solvable, the indices of the variables x_{l,s,o}^{L} that are 1 readily provide the particle species s and orientation o assigned to each lattice position l, allowing visualization of the lattice, as shown in FIG. 1E.

To demonstrate the versatility of the SAT mapping approach for particle design, three of the most challenging and sought after lattice geometries were selected. After using the SAT solver to obtain PP species design and type interaction matrix, molecular simulations were ran and the success and quality of crystals obtained from homogenous nucleation were studied. The SAT solver guarantees that the target structure is an energy minimum, but cannot say whether kinetic traps or other energy minima, both often associated with competing crystalline structures, are present along the self-assembly pathway. If a competing structure is found in the molecular simulations, it can be explicitly excluded by redesigning the SAT problem by adding additional clauses or by discarding all generated solutions that can form the identified undesired structures. Thus, the framework iteratively arrives at a design which self-assembles into the desired crystal formation through homogeneous nucleation. In contrast to previous solutions to this problem, it is stressed that these crystalline structures are being nucleated without introducing torsional interactions or hierarchical assembly. Moreover, the crystals are nucleated homogeneously, without the need for seeding or templating, and grow without stacking defects.

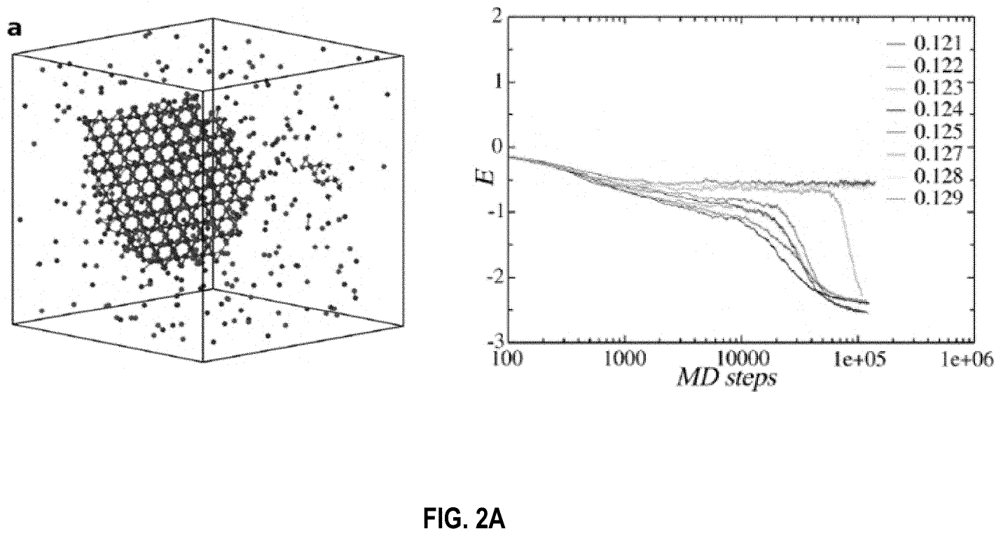

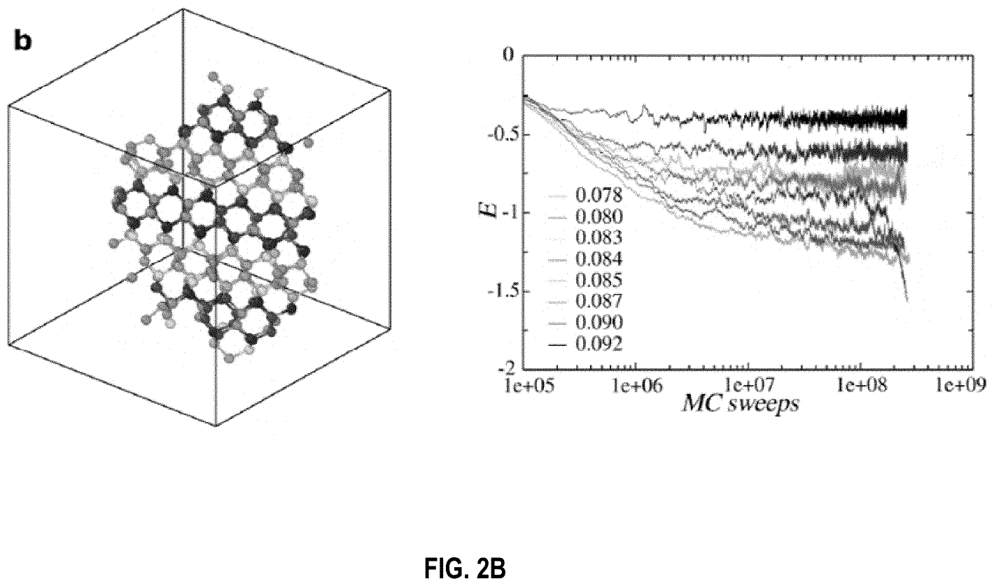

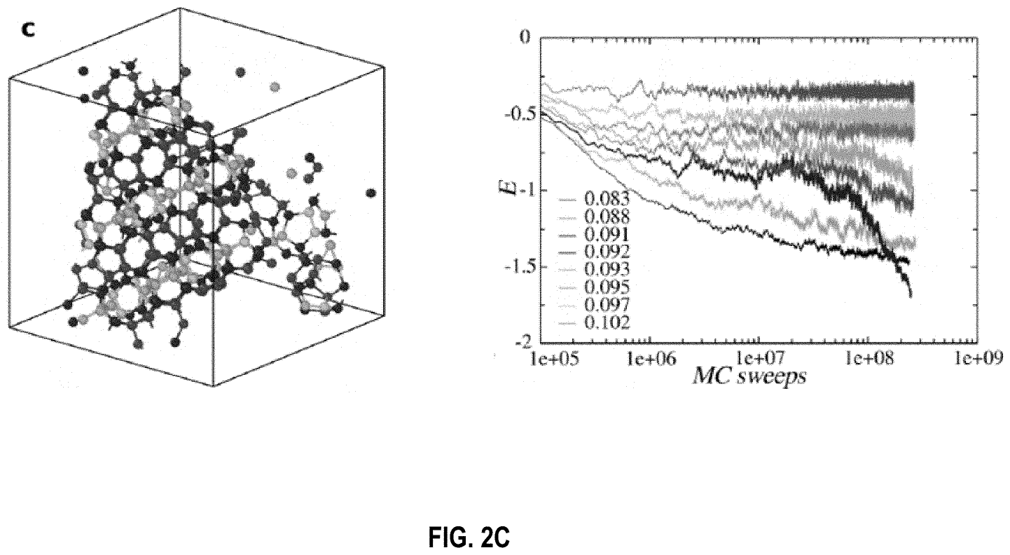

The first target structure is the cubic tetrastack (TS) lattice (also known as pyrochlore, shown in FIG. 2A) that together with the cubic diamond has been proposed for its omnidirectional photonic band gap for use as a photonic crystal. A design with 6 patches in the direction of the closest neighbors is adopted, as shown in FIGS. 1A and 1C. To mimic the possible experimental realization of the system using 3D DNA nanostructures, with single-stranded DNA representing individual patches, the PPs are modeled as soft spheres with attractive point-patches (as described below in “Patchy Particle Models and SAT Solver Logic” section) Solutions can be found with Nₛ=1, N_{c}=3 but suffer from the geometric problem in which two particles can bind with two bonds at the same time, leading to alternative assemblies in the simulations. To avoid this, an additional clause is introduced (further defined in “Patchy Particle Models and SAT Solver Logic” section) that requires that no pair of particles can bind through more than one bond. This SAT problem has no solution (null solution) for Nₛ=1. For Nₛ=2, solutions were found for different N_{c}, and the one with N_{c}=12 was tested in simulation (FIGS. 1D and 1E), which showed successful nucleation (FIG. 2A). To simulate the assembly kinetics, simulations at a range of temperatures of a point-patch particle model were used (further defined in “Patchy Particle Models and SAT Solver Logic” section). The simulations were carried out with 2048 particles at number density 0.1 (corresponding to a volume fraction of 0.05). This density was chosen to mimic the common experimental scenario of phase-separation-induced crystallization from a low-density solution. FIG. 2A shows a nucleation event: the left panel shows a snapshot of the nucleus, the right panel shows the time evolution of the energy for runs at different temperatures, where the nucleating trajectory is signaled by the sharp decrease in energy

Next, consider the tetravalent PP assembly. One of the most popular models for the study of tetravalent systems is the Kern-Frenkel (KF) potential, which is a square-well potential with angular dependence (see “Patchy Particle Models and SAT Solver Logic” section) The thermodynamic and crystallizability of the model have been well characterized, and have highlighted the difficulty of obtaining nucleations of a pure crystal due to the many kinetic traps represented by competing structures. Due to the discontinuous nature of the potential, the Monte Carlo (MC) method is commonly employed to study its assembly. Previous studies have shown the nucleation process to be very similar between MD and MC simulations, because the dynamics in the melt is not significantly different between MD and MC integrators (as long as the MC trial displacements are small).

The assembly of the cubic diamond (DC, shown in FIG. 2B) lattice is further sought, probably the most sought-after crystal for photonic applications. Systems that can assemble DC lattice are almost inevitably found to be also able to assemble into hexagonal diamond (HD) lattice, resulting in imperfect crystals with defects and stacking faults. A tetrahedral PP design (Nₚ=4) was adopted, and solutions that type an 8-particle unit cell of DC but cannot type an 8-particle unit cell of HD were looked for. Even in this case, the SAT solver showed that any PP solution that satisfies 8-particle DC cell can also assemble a 32-particle HD unit cell. A strategy to avoid the HD lattice in this case is to employ a larger unit cell for the DC. Hence, a larger 16-particle unit cell of a DC lattice was used and a range of combinations of Nₛ>8 and N_{c} were scanned. For each solution that was obtained for a given Nₛ and N_{c}, each solution was checked with the SAT solver to determine if it can assemble into a 32-particle HD unit cell. The solution with the smallest Nₛ that was found to be able to form DC and not form 32-particle HD cell had Nₛ=9 and N_{c}=31, and it successfully nucleated the DC lattice in a Kern-Frenkel PP simulation (FIG. 2B), which was done with 495 particles split equally into 9 PP species, at number density 0.2 (corresponding to volume fraction 0.1). Simulations were also carried out at 0.1 number density with 2048 particles, which also showed homogeneous nucleation of a DC lattice (further discussed in “Patchy Particle Models and SAT Solver Logic” section).

As the last example, the present approach was used to find a system able to nucleate into the Si34 clathrate (CSi34, shown in FIG. 2C) starting from tetrahedral PPs in the KF model. The smallest Nₛ for which a solution was found that could form CSi34 and not DC or HD lattices had Nₛ=4, N_{c}=12, and was confirmed to successfully nucleate CSi34 (FIG. 2C) in a simulation at 0.2 number density with 476 particles in species ratio 6:3:2:6, as well as in a larger simulation at number density 0.1 and 1904 particles (shown in “Patchy Particle Models and SAT Solver Logic” section).

The patch typing and interaction matrices for all PP solutions are given in the Supp. Mat. While the framework has focused so far only on designing systems for the assembly of difficult 3D lattices, the approach can be generalized to other systems, such as finite-size clusters. The framework is not limited to spherical PPs and can be used for any model where simulations or other stochastic methods can identify undesired assemblies as a list of interactions between PP species and their patches. It can be also combined with other techniques, such as using different strengths of interactions to disfavor undesired assemblies identified in the simulations. The approach proposed here is extensible and the systems designed in the present system should be amenable of experimental realization.

### Patchy Particle Models and SAT Solver Logic

The patchy particle (PP) models that were used to carry out the simulations of the lattice assemblies are further discussed herein. Both 6-valent PP model for TS assembly and tetrahedral PP model for DC and CSi34 lattice assembly were implemented in the oxDNA simulation tool package and are freely available with its source code. The energy scale is in simulation unit energy ∈*, and the simulation temperature reported in FIGS. 2A-2C is in reduced units ∈*/kB. For each simulated system, a few simulations were run at empirically chosen temperatures. A temperature range was identified such that at the lowest temperature, the PPs would form a glassy state, and at the highest temperature they would remain in the gas state. The range was then partitioned into different temperatures at 0.001 interval to run respective simulations to find optimal crystallization temperature. Each simulation was started from randomly positioned non-overlapping PPs.

### Patchy Particle Model for Tetrastack Assembly

In simulations of tetrastack (TS) lattice assembly of FIG. 2A, each particle is represented by a sphere covered by 6 patches at distance dₚ=0.5 distance units (d.u.) from the center of the sphere. The positions of the patches, defined in terms of the orthonormal base associated with the patchy particle, are:

p₁=α(0,1,ξ)

p₂=α(0,−1,ξ)

p₃=α(ξ,0,1)

p₄=α(0,−1,−ξ)

p₅=α(0,1,−ξ)

p₆=α(−ξ(0,−1),

where ξ=(1+\sqrt{5})/2 and α=dₚ/\sqrt{1+ξ2}. The position of patch i in the simulation box coordinate system is given by

rₚ_{ᵢ}=r_{cm}+pᵢ_{ₓ},e₁+pᵢ_{_{y}}e₂+pᵢ_{_{z}}e₃ (S1)

where r_{cm} is the center of mass of the patchy particle, and e_{1,2,3} are the x, y, and z orthonormal base vectors associated with the patchy particle's orientation

The interaction potential between a pair of patches on two distinct particles is

$${V}_{patch} ( {r}_{p} ) = \{ \begin{matrix}- 1.001 {δ}_{ij} exp [ - {( \frac{{r}_{p}}{α} )}^{10} ] - C & if {r}_{p} \leq {r}_{pmax} \\ 0 & otherwise\end{matrix} \qquad ( S 2 )$$

where δᵢⱼ is 1 if patch types i and j can bind and 0 otherwise, rₚ is the distances between a pair of patches, and α=0.12 d.u. sets the patch width. The constant C is set so that for V_{patch}(rₚₘₐₓ)=0, rₚₘₐₓ=0.18 d.u. The patchy particles further interact through excluded volume interactions ensuring that two particles do not overlap

$${f}_{exc} ( r , ϵ , σ , {r}^{*} ) = \{ \begin{matrix}{V}_{LJ} ( r , ϵ , σ ) & if r < {r}^{*} , \\ ϵ {V}_{smooth} ( r , b , {r}^{c} ) & if {r}^{*} < r < {r}^{c} , \\ 0 & otherwise .\end{matrix} \qquad ( S 3 )$$

where r is the distance between the centers of the patchy particles, and σ is set to 2R=0.8, twice the desired radius of the patchy particle. The choice of a radius smaller than dₚ has been done to mimic very soft particles, where the assembly has to be driven purely by bonds without being affected by excluded volume. The repulsive potential is a piecewise function, consisting of Lennard-Jones potential function

$${V}_{LJ} ( r , σ ) = 8 [ {( \frac{σ}{r} )}^{12} - {( \frac{σ}{r} )}^{6} ] . \qquad ( S 4 )$$

that is truncated using a quadratic smoothening function

Vₛₘₒₒₜₕ(x,b,x^{c})=b(x_{c}−x)², (S5)

with b and x_{c} are set so that the potential is a differentiable function that is equal to 0 after a specified cutoff distance r^{c}=0.8.

The patchy particle system was simulated using rigid-body Molecular Dynamics with an Andersen-like thermostat as well as using Monte Carlo (MC) simulations. During the simulation, each patch was only able to be bound to one other patch at the time, and if the binding energy between a pair of patches, as given by Eq. (S2), is smaller than 0, none of the patches can bind to any other patch until their pair interaction potential is again 0.

### Patchy Particle Model for Diamond and Clathrate Lattice Assembly

For the diamond cubic (DC) and clathrate Si34 (CSi34) lattice assembly of FIGS. 2B and 2C, tetravalent patchy particles with tetrahedral patch arrangements are used. The positions of the patches, in the orthonormal base associated with the patchy particle, are given as

p₁=R(\sqrt{8/9},0,−⅓)

p₂=R(−\sqrt{2/9},\sqrt{⅔},−⅓)

p₃=R(−\sqrt{2/9},−\sqrt{⅔},−⅓)

p₄=R(0,0,1),

where R=0.5 d.u. is the radius of the patchy particle represented by a sphere. MC simulations of the DC and CSi34 lattice assembly is performed. Each patchy particle is modeled as a hard sphere, with excluded volume interaction between two particles at distance r defined as

$${V}_{hs} ( r ) = \{ \begin{matrix}∞ & if r < 2 R , \\ 0 & otherwise .\end{matrix} \qquad ( S 6 )$$

The interaction between a pair of patches pᵢ and qⱼ on distinct particles i and j is modeled through the Kern-Frenkel interaction potential:

$${V}_{FK} ( r , {θ}_{p} , {θ}_{q} ) = \{ \begin{matrix}- 1 & if r < 2 R + δ and cos {θ}_{p} < {θ}_{max} and cos {θ}_{q} < {θ}_{max} , \\ 0 & otherwise .\end{matrix} \qquad ( S 7 )$$

where δ is set to 0.12 d.u. in our simulations and θₘₐₓ is set to 0.98. Furthermore, we use r=r_{cm}_{_{q}}r_{cm}_{ₚ}, where r=∥r∥ is the distance between the centers of mass of the patchy particles p and q, to define angles

$$cos {θ}_{p} = \frac{r \cdot {p}_{i}}{‖ r ‖ ‖ {p}_{i} ‖} \qquad ( S 8 )$$

$$cos {θ}_{q} = \frac{- r \cdot {q}_{j}}{‖ r ‖ ‖ {q}_{j} ‖} \qquad ( S 9 )$$

where pᵢ is the vector from center of mass of particle p towards patch pᵢ, and analogously for patch qⱼ.

### Simulations of DC and CSi34 Lattice Assembly at 0.1 Number Density

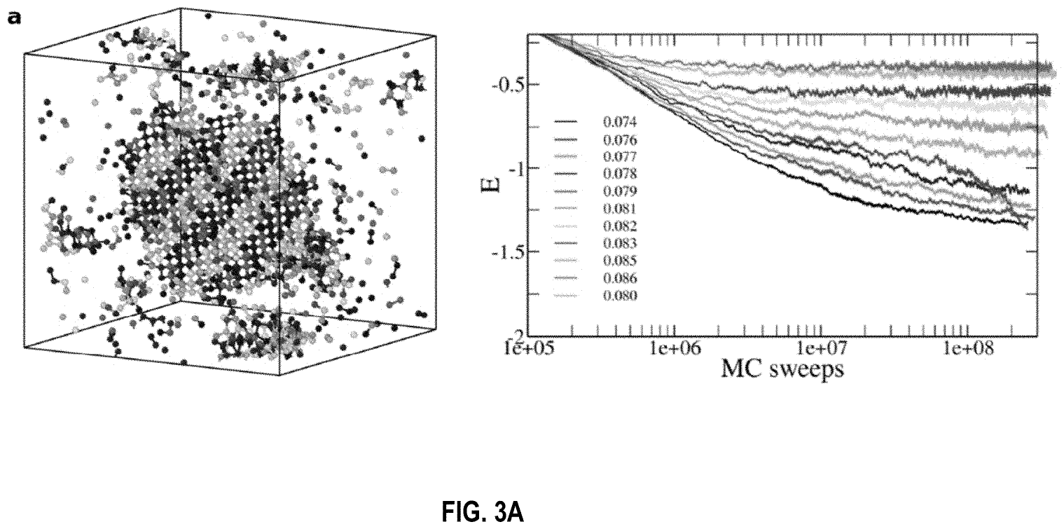

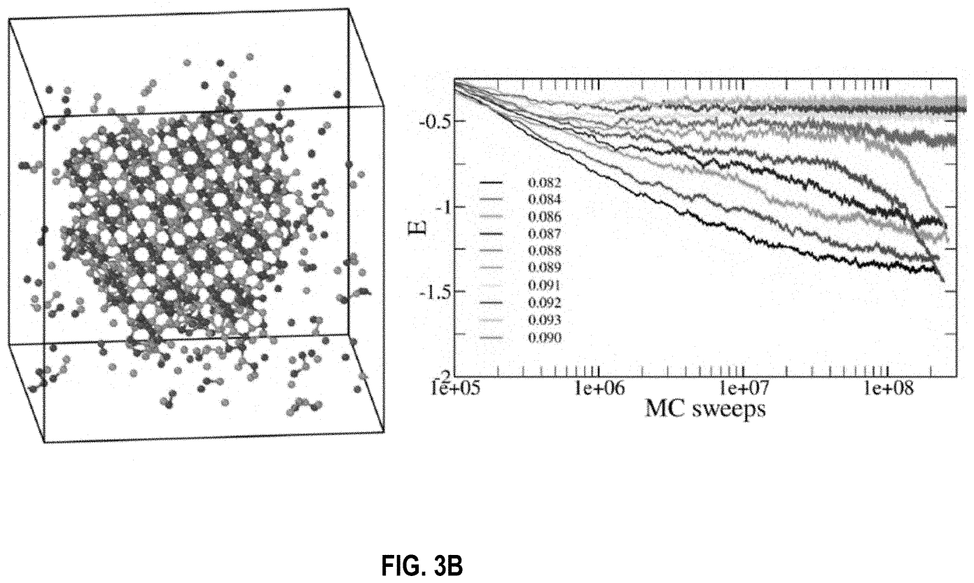

Additionally to simulations shown in FIGS. 2A-2C, further simulations include the homogeneous nucleation of the DC lattice with a simulation 2048 particles (at 0.1 number density, corresponding to 0.05 volume fraction), split into the 9 respective species in ratio 2:2:2:1:2:2:2:1:2, which corresponds to the number of times each PP species is represented in the 16-particle DC unit cell. CSi34 lattice assembly was further simulated with 1904 particles respectively, split into 4 species in ratio 6:3:2:6, corresponding to the relative PP species ratio in the 34-particle unit cell of CSi34. Both systems have shown homogeneous nucleation and the summary of the simulations is in FIG. 3A.

### Patchy Particle Solutions for Crystal Lattices

Listed here are the patch typing and type interaction rules that were found by the SAT solver and verified by simulations to assemble into the TS, DC, and CSi34 lattices respectively. For each PP species, the patch typings are specified as a list (p, c), where p is a number that identifies the patch on a particle (1 to 6 for patchy particles that assemble in TS lattice, and 1 to 4 for patchy particles that assemble in DC or CSi34 lattice), and c identifies the patch type (from 1 to N_{c}, where N_{c} is the total number of types used). The interacting types are then listed as pairs (cᵢ, cⱼ), where type cᵢ can only bind to type cⱼ and vice versa. For self-complementary types, list (cᵢ, cⱼ). The designs of patchy particles for assembly of TS, DC, and CSi34 lattices respectively are given below:

TS crystal lattice design with Nₛ=2 and N_{c}=12:

| PP species | Patch Coloring | | | | | |
|---|---|---|---|---|---|---|
| 1: | (1, 1) | (2, 2) | (3, 3) | (4, 4) | (5, 5) | (6, 6) |
| 2: | (1, 7) | (2, 8) | (3, 9) | (4, 10) | (5, 11) | (6, 12) |

Color interactions
(1, 5), (2, 12), (3, 8), (4, 7), (6, 11), (9, 10)

DC crystal design with Nₛ=9 and N_{c}=31:

| | |
|---|---|
| PP species | Patch Coloring |

| | | | | |
|---|---|---|---|---|
| 1: | (1, 16) | (2, 9) | (3, 18) | (4, 4) |
| 2: | (1, 26) | (2, 13) | (3, 23) | (4, 1) |
| 3: | (1, 5) | (2, 8) | (3, 24) | (4, 31) |
| 4: | (1, 12) | (2, 21) | (3, 27) | (4, 19) |
| 5: | (1, 12) | (2, 29) | (3, 14) | (4, 17) |
| 6: | (1, 28) | (2, 15) | (3, 7) | (4, 6) |
| 7: | (1, 3) | (2, 22) | (3, 11) | (4, 2) |
| 8: | (1, 21) | (2, 30) | (3, 12) | (4, 19) |
| 9: | (1, 20) | (2, 25) | (3, 8) | (4, 10) |

Color interactions
(1, 4), (2, 25), (11, 17), (12, 12), (13, 13), (6, 18)
(16, 31), (19, 22), (20, 23), (3, 9), (5, 26), (7, 14)
(21, 28), (24, 24), (27, 30), (29, 29), (8, 8), (10, 15)

CSi34 crystal design with Nₛ=4 and N_{c}=12:

| | |
|---|---|
| PP species | Patch Coloring |

| | | | | |
|---|---|---|---|---|
| 1: | (1, 3) | (2, 11) | (3, 8) | (4, 5) |
| 2: | (1, 12) | (2, 9) | (3, 4) | (4, 12) |
| 3: | (1, 7) | (2, 7) | (3, 4) | (4, 7) |
| 4: | (1, 6) | (2, 10) | (3, 1) | (4, 2) |

Color interactions
(1, 1) (2, 2) (3, 12) (4, 4) (5, 6) (7, 8) (9, 9) (10, 11)

### Boolean Clause Formulation for SAT

The PP design problem is formulated as a SAT problem by specifying it as a set of binary clauses in a format acceptable by SAT solver software. A detailed formulation is provided of the binary clauses in a format required as an input into the commonly used SAT solvers

The set of binary clauses as defined in Table 1 fully specify the PP design problem. For a target lattice structure with unit cell consisting of L positions, where each position has Nₚ neighbors (and hence Nₚ slots), we are looking for a solution that has Nₛ PP species and uses in total Nₚ different types. The particle geometry (i.e. patch positions on the PP) is hard-coded by the lattice geometry, and we need to provide all possible rotations No that a PP can make in a given lattice position so that its patches overlap with the slots of the given position (illustrated in FIGS. 1A-1E). For the PPs used for TS lattice assembly Nₒ=6. For tetrahedral PPs used in DC and CSi34 lattices, Nₒ=12. For each PP orientation o, we assign a mapping (ϕ₀, which maps patch on the PP to the slot of the lattice position, e.g. (ϕ₁=(1,2,3,4,5,6)→(2,3,1,5,6,4) for a particle with 6 patches used for TS assembly.

Binary variables x_{c}_{ᵢ}_{,c}_{ⱼ}^{int} are defined, which are 1 if given pair cᵢ≤cⱼ∈[1,N_{c}] of types can interact and 0 if they cannot. Next set of variables x_{s,p,c}^{pcol} define typing of patches for each particle species s∈[1,Nₛ] and x_{s,p,c}^{pcol} is 1 if p-th patch (p∈[1,Nₚ]) of s-th particle species is assigned to have type c. A set of variables that define arrangement of PP types in the lattice are further defined, where x_{l,s,r}^{L} is 1 if in the desired lattice geometry, the PP species that occupies position l∈[1,L] is of PP type ^{s}∈[1,Nₛ] and its orientation is set to o∈[1,Nₒ]. Lastly, variables x_{l,k,c}^{A} are defined, which are 1 if the PP in lattice position l∈[1,L] is oriented in such a way that the slot k∈[1,Nₚ] of that position overlaps with PP's patch that has type c. The variables are defined for all possible combinations of types, PP species, patches, orientations, lattice positions. The solution is specified by a list of variables that are 1 (true). For instance x_{c}_{ₐ}_{,c}_{_{b}}^{int}=1 means that in the type interaction matrix, type cₐ is compatible with c_{b}. However, to make sure that the assignment of true and false values to all defined variables is a correct solution to the design task, it is necessary to define binary clauses that introduce relations between the variables that have to be satisfied.

The clauses (i)-(iii) in Table 1 ensure feasibility of the solution: as binary variables x^{pcol}, x^{L}, x^{int} correspond to all possible combinations of type interactions, patch typing and PP assignment to positions on the lattice, the first three sets of clauses ensure that in the obtained solution, the variables that are mutually exclusive (e.g. a patch having at the same time two different types) cannot be both true at the same time. The clauses (iv)-(v) enforce that for the correct solution, the PPs have to be arranged in the target lattice in such a way that patches in contact have compatible types. Finally, clauses (vi)-(vii) impose that for the solution found by the SAT solver, all Nₛ PP species have to be included in the lattice formation, and all N_{c} types have to be used. This requirement is added to avoid the solvers coming up with trivial solutions, such as designing one PP species with all patches typed to a self-complementary type, which would then trivially satisfy the target lattice.

As an input for the SAT solver, the problem has to be formulated in terms of clauses Cⱼ, each of them containing variables xᵢ connected by OR clauses. The final SAT problem corresponds to all respective clauses Cⱼ connected by AND clauses. To conform with this input format, the Boolean clauses introduced in Table 1 in the main text can be all reformulated as detailed below. The final SAT problem is a conjunction of all individual clauses from sets (i)-(vii) described below. The definitions use the following logic symbols: ¬: negation; ∧: conjunction (AND); ∨: disjunction (OR); ⇒: implies; ⇐ ⇒: if and only if.

(i) Each type ci can only bind to one other type C_{f} (including possible self-complementarity).

∀cᵢ≤cⱼ<cₖ∈[1,N_{c}]:C_{c}_{ᵢ}_{,c}_{ⱼ}_{,c}_{ₖ}^{int}=¬x_{c}_{ᵢ}_{,c}_{ⱼ}^{int}∧¬x_{c}_{ᵢ}_{,c}_{ₖ}^{int}. (S10)

To illustrate how the above clauses achieve unique binding int between types, consider variable x_{c}_{ₐ}_{,c}_{_{b}}^{int}=1 for particular choice of cₐ and c_{b} (meaning that these types can bind). Consider a type cₖ different from cₐ and c_{b}. The SAT problem includes conjunction of all clauses C^{int} as defined in Eqs. S10. Hence all of the clauses have to be true, including the clause C_{c}_{ₐ}_{,c}_{_{b}}_{,c}_{ₖ}^{int}=¬x_{c}_{ₐ}_{,c}_{_{b}}^{int}∧¬x_{c}_{ₐ}_{,c}_{ₖ}^{int}. Since ¬x_{c}_{ₐ}_{,c}_{_{b}}^{int} is false, satisfying this clause is only possible if x_{c}_{ₐ}_{,c}_{ₖ}^{int}=0, i.e. types cₐ and cₖ are not allowed to interact. Analogously, it can be shown that x_{c}_{_{b}}_{,c}_{ₖ}^{int} must be 0 and therefore c_{b} and cₖ do not interact either.

(ii) Each patch p of each PP species s is assigned exactly one type:

∀s∈[1,Nₛ],p∈[1,Nₚ],cₗ<cₖ∈[1,N_{c}]:C_{s,p,c}_{ₖ}_{,c}_{ₗ}^{pcol}=¬x_{s,p,c}_{ₖ}^{pcol}∧¬x_{s,p,c}_{ᵢ}^{pcol}. (S11)

In a manner analogous to Eqs. S10, the clauses C^{pcol} ensure that if for example x_{s,p,c}_{ₐ}^{pcol} is 1, the framework needs to have x_{s,p,c}_{ₖ}^{pcol}=0 for all other cₖ≠0 ca in order to satisfy the C^{pcol} clauses, and hence patch p on PP species s can only have type cₐ.

(iii) Each lattice position l is only assigned exactly one PP species with exactly one assigned orientation:

∀l∈[1,L],sᵢ<sⱼ∈[1,Nₛ],oᵢ<oⱼ∈[1,Nₒ]:C_{l,s,o}_{ᵢ}_{,s}_{ⱼ}_{,o}_{ⱼ}^{L}=¬x_{l,s}_{ᵢ}_{,o}_{ᵢ}^{L}∧¬x_{l,s}_{ⱼ}_{,o}_{ⱼ}^{L}. (S12)

Analogously to clauses (i) and (ii), the set of clauses defined in Eqs. S12 ensure that there can be only one PP species with only one assigned orientation occupying given slot I in the target lattice.

(iv) For all pairs of slots kᵢ and kⱼ that are in contact in neighboring lattice positions lᵢ, lⱼ (e.g. as shown in FIG. 1B), the patches that occupy them need to have complementary types:

∀cᵢ≤cⱼ∈[1,N_{c}]:Cₗ_{ᵢ}_{,k}_{ᵢ}_{,l}_{ⱼ}_{,k}_{ⱼ}_{,c}_{ᵢ}_{,c}_{ⱼ}^{lint}=(xₗ_{ᵢ}_{,k}_{ᵢ}_{,c}_{ᵢ}^{A}∧xₗ_{ⱼ}_{,k}_{ⱼ}_{,c}_{ⱼ}^{A})⇒x_{c}_{ᵢ}_{,c}_{ⱼ}^{C},

which can be equivalently rewritten as

Cₗ_{ᵢ}_{,k}_{ᵢ}_{,l}_{ⱼ}_{,k}_{ⱼ}_{,c}_{ᵢ}_{,c}_{ⱼ}^{lint}=¬xₗ_{ᵢ}_{,k}_{ᵢ}_{,c}_{ᵢ}^{A}∨¬xₗ_{ⱼ}_{,k}_{ⱼ}_{,c}_{ⱼ}^{A}∨x_{c}_{ᵢ}_{,c}_{ⱼ}^{C}. (S13)

These clauses assure that PPs placed in neighboring positions in the lattice interact through the correctly typed slots. The Eqs. (S13) hence encode the geometry of the target lattice.

(v) The slot of lattice position I is typed with the same type as the patch of the PP species occupying it:

∀l∈[1,L],k∈[1,Nₚ],o∈[1,Nₒ],s∈[1,Nₛ],c∈[1,N_{c}]:C_{l,s,o,c,k}^{LS}=x_{l,s,o}^{L}⇒(x_{l,k,c}^{A}⇔x_{s,ϕ}_{ₒ}_{(k),c}^{pcol}),

which can be equivalently rewritten as

C_{l,x,o,c,k}^{LS}=(¬x_{l,s,l}^{L}∨¬x_{l,k,c}^{A}∨x_{s,ϕ}_{ₒ}_{(k),c}^{pcol})∧(¬x_{l,s,l}^{L}∨x_{l,k,c}^{A}≙¬x_{s,ϕ}_{ₒ}_{(k),c}^{pcol}) (S14)

These clauses are required to correctly set variables x^{A}, which are used in clauses (iv). A PP of type s can be placed in Nₒ different orientations o into a specific position l on the lattice. For a particular choice of o, the mapping function φₒ maps φₒ(k)-th patch to k-th slot, assuring that variable x_{l,k,c}^{A} is 1 if the φₒ(k)-th patch of particle on lattice position l has type c.

(vi) All Nₛ PP species have to be used at least once in the assembled lattice:

$$∀ s ∈ [ 1 , {N}_{s} ] : {C}_{s}^{all s .} = {∨}_{∀ l ∈ [ 1 , L ] , o ∈ [ 1 , {N}_{o} ]} {x}_{l , s , o}^{L} \qquad ( S 15 )$$

For each s, the variables in the clause Cₛ^{alls} are connected by OR (disjunction). Each clause contains al combinations of variables for all lattice positions l and orientations o. These clauses ensure that in the solution, each PP species appears at least once in the lattice. For examples, consider that there is a solution that does not contain PP species number sa in the lattice. In that case, variables x_{l,s}_{ₐ}_{,o}^{L} are 0 for all values of l and o, which makes the clause Cₛ_{ₐ}^{alls.} false.

(vii) Each type c of N_{c} total number of types is assigned to at least one patch of one of the PP species

$$∀ c ∈ [ 1 , {N}_{c} ] : {C}_{c}^{all c .} = {∨}_{∀ s ∈ [ 1 , {N}_{s} ] , p ∈ [ 1 , {N}_{p} ]} {x}_{s , p , c}^{pcol} . \qquad ( S 16 )$$

These clauses ensure that all types are used in the solution in a similar way that clauses (vi) ensure that all PP species are used.

For the design of the TS lattice, we introduced an additional set of clauses that ensure that any pair of particles (of the same or different species) cannot bind by more than one bond at a time:

∀sᵢ,sⱼ∈[1,Nₛ],cᵢ¹,cᵢ²,cⱼ¹,cⱼ²∈[1,N_{c}]C_{s,s}_{ⱼ}_{,p}_{₁}_{ⁱ}_{,p}_{₂}_{ⁱ}_{,p}_{₁}_{ⁱ}_{,p}_{₂}_{^{j}}_{,c}_{ᵢ}_{¹}_{,c}_{ᵢ}_{²}_{,c}_{ⱼ}_{¹}_{,c}_{ⱼ}_{²}=¬(x_{s,p}_{₁}_{ⁱ}_{,c}_{ᵢ}_{¹}^{pcol}∧xₛ_{ᵢ}_{,p}_{₂}_{ⁱ}_{,c}_{ᵢ}_{²}^{pcol}∧xₛ_{ⱼ}_{,p}_{₁}_{ⁱ}_{,c}_{ⱼ}_{¹}^{pcol}∧xₛ_{ⱼ}_{,p}_{₂}_{ⁱ}_{,c}_{ⱼ}_{²}^{pcol}∧x_{c}_{ᵢ}_{¹}_{,c}_{ⱼ}_{¹}^{int}∧x_{c}_{₂}_{ⁱ}_{,x}_{ⱼ}_{²}^{int}), (S17)

where p₁ⁱ, p₂ⁱ, p₁^{j}, p₂^{j} are all possible pairs of patches on PP of type sᵢ and sⱼ respectively for which there is a possible orientation so that they can both bind if they have compatible types.

To find solutions for SAT problems considered in the present disclosure, MiniSat, MapleSAT, or Walksat solvers were used. These are popular standard tools used by researchers in constraint satisfaction problems community and we used them as ‘black box’. Walksat was found to be the fastest in finding the solutions to most of the PP design problems (typically in several seconds). However, if the solution does not exist, Walksat algorithm is unable to prove the impossibility, as it just continues its search for a solution even if it does not exist. MapleSAT had performance similar to MiniSat in terms of time it took them to find a solution (between few seconds to tens of minutes), but MapleSAT is more memory efficient (less than 2 GB RAM for SAT problems that we encountered in this work), allowing multiple MapleSAT solvers to run in parallel without running out of available memory on a typical workstation. As opposed to Walksat, MiniSat and MapleSAT can also be used to prove that no solution (null solution) exists for a given SAT problem. However, for certain combinations of Ns and Nc for DC and CSi34 lattices, the algorithms were not able to find solution nor prove impossibility within the maximum 2 hours running time that was imposed for the solvers. It is possible the solutions could be found (or impossibility proved) if algorithms were run for longer.

For each successful solution that was found for a given lattice and Nₛ and N_{c}, the solution was tested for its ability to assemble into undesired structures using MiniSat. In this case, true values were explicitly set to the combination of patch typing and type interactions (x^{int} and ^{xpcol}) variables that encodes the solution that were found, and use clauses (i)-(v) to formulate the SAT problem. For this task, it takes MiniSat (or MapleSat) only few seconds to find out if the PP species (or their subset) can or cannot assemble into a given undesired lattice. Hence, one can test the solution very quickly against a list of known undesired structures.

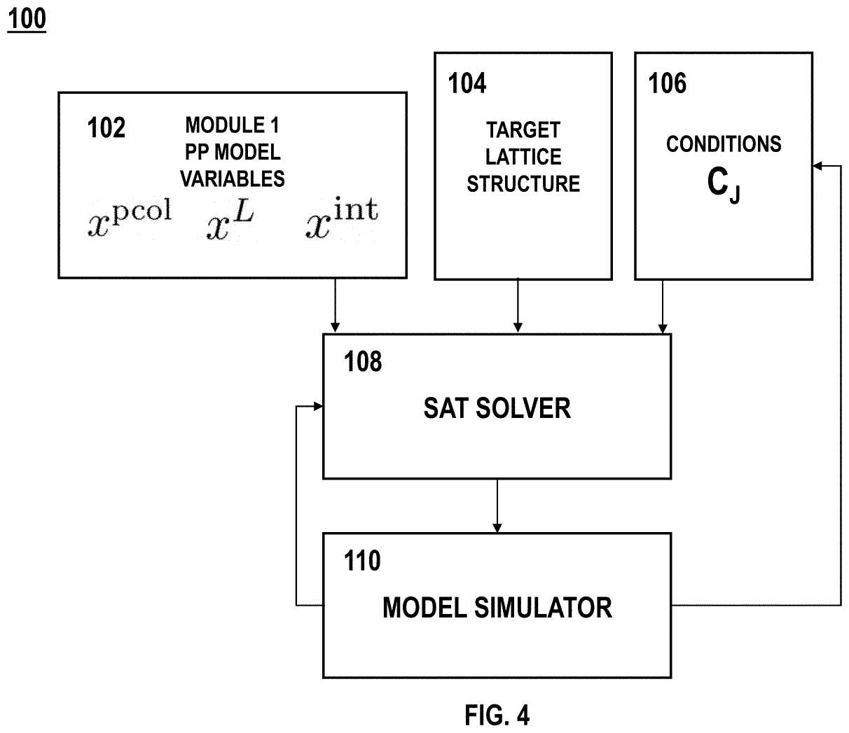

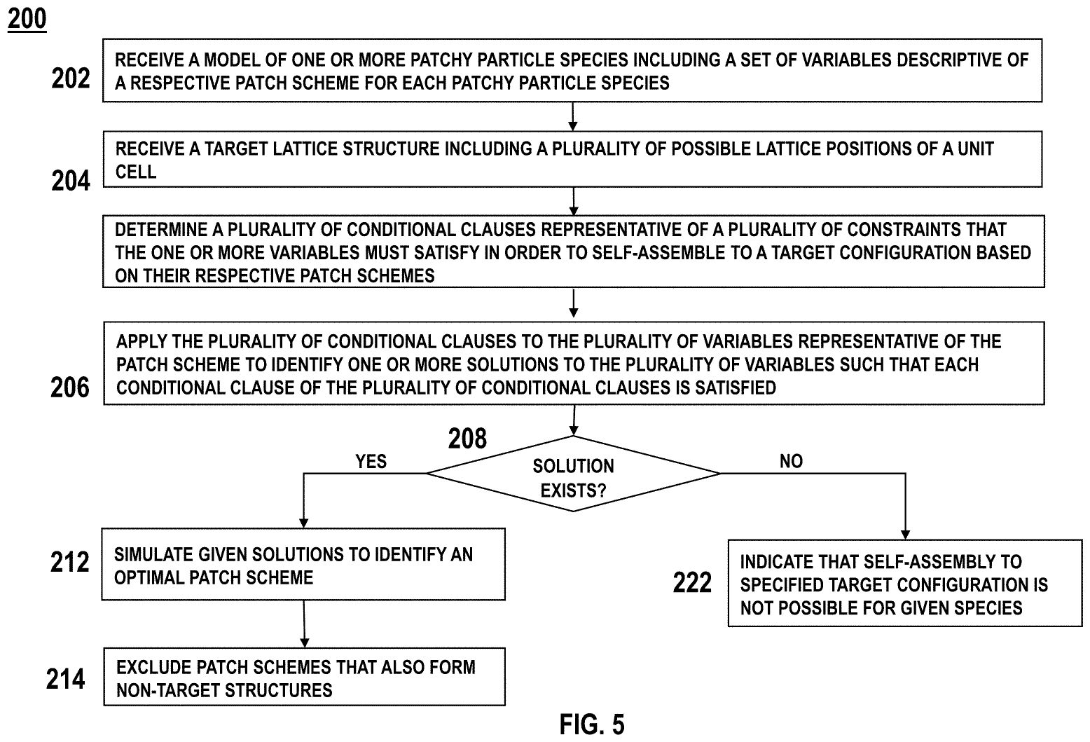

Referring to FIGS. 4 and 5, a general framework 100 and associated methodology 200 for determining a patch type scheme for at least one particle species of patchy particles such that the patchy particles assemble into a target structure.

In particular, FIG. 4 illustrates the framework 100 for determining a solution in the form of a patch type scheme x^{pcol}, an interaction matrix x^{int} and an orientation x^{L} of each PP within a target lattice structure. The framework 100 includes a first module 102 associated with the PP models for each PP species and associated attributes, including definitions for variables corresponding to patch type scheme x^{pcol}, an interaction matrix x^{int} and an orientation x^{L} of each PP within a target lattice structure. Framework 100 can also include a second module 104 associated with the target lattice structure and can include one or more descriptors of a unit cell including one or more target lattice slots. Actions performed by module 102 of the framework 100 corresponds with step 202 of method 200 of FIG. 5, which indicates receiving a model of one or more patchy particle species including the set of variables descriptive of a respective patch scheme for each patchy particle species. Step 204 corresponds to module 104 of framework 100, which indicates receiving a target lattice structure including a plurality of possible lattice positions of a unit cell.

Framework 100 of FIG. 4 can also include a third module 106 including a plurality of conditions to be imposed by an SAT solver such that attributes of the PP models as determined by the SAT solver allow assembly of the plurality of PPs into the target lattice structure. Conditions and associated definitions can include clauses described in Table 1 of this disclosure, but can also be manually added to or improved upon when imposing additional restrictions to prevent formation of certain undesirable lattice structures including competing structures or unstable structures. Actions performed by module 106 of the framework 100 correspond with step 206 of method 200 of FIG. 5, which indicates determining a plurality of conditional clauses representative of a plurality of constraints that the one or more variables must satisfy in order to self-assemble to a target configuration based on their respective patch schemes.

SAT solver module 108 of framework 100 of FIG. 4 receives the PP model definitions from first module 102, target lattice structure definitions from second module 104 and conditions from third module 106 to determine appropriate PP model values including the patch type scheme x^{pcol}, interaction matrix x^{int} and orientation x^{L} of each PP within the target lattice structure such that the PPs assemble into the target lattice structure. Actions performed by SAT solver module 108 of the framework 100 correspond with step 208 of method 200 of FIG. 5, which indicates applying the plurality of conditional clauses to the plurality of variables representative of the patch scheme to identify one or more solutions to the plurality of variables such that each conditional clause of the plurality of conditional clauses is satisfied. In some cases, multiple solutions exist that can be simulated in a further step to select a subset of solutions that produce a desired result. In other cases, a “null” solution is found, corresponding with step 222 of FIG. 5, indicating that no solution exists that will allow assembly into the target structure given one or more parameters of the PP model.

Once SAT solver module 108 identifies one or more solutions including one or more patch type schemes, interaction matrices, and orientations of each PP within the target lattice structure, a model simulator module 110 of FIG. 4 can be used to verify the one or more solutions. In particular, simulating the solutions by model simulator module 110 can identify solutions that also create competing or otherwise undesirable lattice structures. Such solutions can then be removed from a listing of viable solutions, and in some embodiments, additional conditional clauses can be added to module 106 to provide further preventative limitations when running the SAT solver module 108 to identify solutions. Actions performed by model simulator module 110 of the framework 100 correspond with step 212 of method 200 of FIG. 5, which indicates simulating given solutions to identify an optimal patch scheme for each of the one or more patchy particle species. Step 214 indicates excluding patch schemes or solutions that form undesirable lattice structures.

### Computer-Implemented System

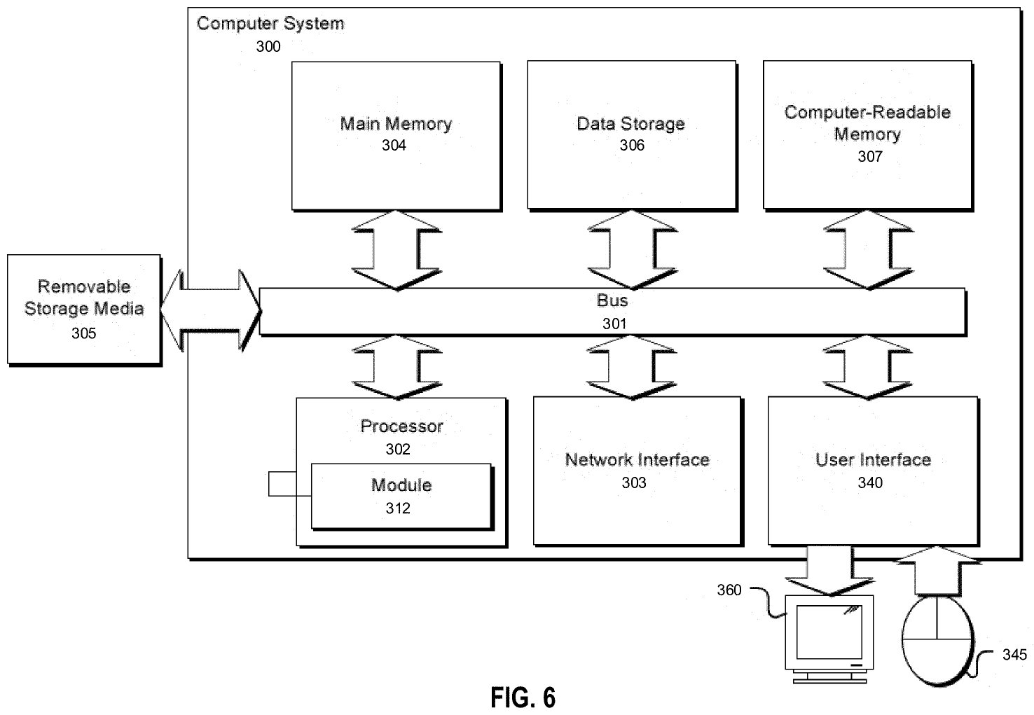

FIG. 6 illustrates an example of a suitable computing and networking environment (computer system 300) which may be used to implement various aspects of the present disclosure. Example embodiments described herein may be implemented at least in part in electronic circuitry; in computer hardware executing firmware and/or software instructions; and/or in combinations thereof. Example embodiments also may be implemented using a computer program product (e.g., a computer program tangibly or non-transitorily embodied in a machine-readable medium and including instructions for execution by, or to control the operation of, a data processing apparatus, such as, for example, one or more programmable processors or computers). A computer program may be written in any form of programming language, including compiled or interpreted languages, and may be deployed in any form, including as a stand-alone program or as a subroutine or other unit suitable for use in a computing environment. Also, a computer program can be deployed to be executed on one computer, or to be executed on multiple computers at one site or distributed across multiple sites and interconnected by a communication network.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software, in the form of the system application 200 or otherwise, may include a hardware-implemented module and may accordingly configure a processor 302, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

As illustrated, the computing and networking environment 300 may be a general purpose computing device 300, although it is contemplated that the networking environment 300 may include other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the general purpose computing device 300 may include various hardware components, such as a processing unit 302, a main memory 304 (e.g., a memory or a system memory), and a system bus 301 that couples various system components of the general purpose computing device 300 to the processing unit 302. The system bus 301 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The general purpose computing device 300 may further include a variety of computer-readable media 307 that includes removable/non-removable media and volatile/nonvolatile media, but excludes transitory propagated signals. Computer-readable media 307 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EPSOM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the general purpose computing device 300. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 304 includes computer storage media in the form of volatile/nonvolatile memory such as read only memory (ROM) and random access memory (RAM). A basic input/output system (BIOS), containing the basic routines that help to transfer information between elements within the general purpose computing device 300 (e.g., during start-up) is typically stored in ROM. RAM typically contains data and/or program modules that are immediately accessible to and/or presently being operated on by processing unit 302. For example, in one embodiment, data storage 306 holds an operating system, application programs, and other program modules and program data.

Data storage 306 may also include other removable/non-removable, volatile/nonvolatile computer storage media. For example, data storage 306 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules and other data for the general purpose computing device 300.

A user may enter commands and information through a user interface 340 or other input devices 345 such as a tablet, electronic digitizer, a microphone, keyboard, and/or pointing device, commonly referred to as mouse, trackball, or touch pad. Other input devices 345 may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices 345 are often connected to the processing unit 302 through a user interface 340 that is coupled to the system bus 301, but may be connected by other interface and bus structures, such as a parallel port, game port or a universal serial bus (USB). A monitor 360 or other type of display device is also connected to the system bus 301 via user interface 340, such as a video interface. The monitor 360 may also be integrated with a touch-screen panel or the like.

The general purpose computing device 300 may operate in a networked or cloud-computing environment using logical connections of a network Interface 303 to one or more remote devices, such as a remote computer. The remote computer may be a personal computer, a server, a router, a network PC, a peer device or other common network node, and typically includes many or all of the elements described above relative to the general purpose computing device 300. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the general purpose computing device 300 may be connected to a public and/or private network through the network interface 303. In such embodiments, a modem or other means for establishing communications over the network is connected to the system bus 301 via the network interface 303 or other appropriate mechanism. A wireless networking component including an interface and antenna may be coupled through a suitable device such as an access point or peer computer to a network. In a networked environment, program modules depicted relative to the general purpose computing device 300, or portions thereof, may be stored in the remote memory storage device.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method for determining a patch type scheme for at least one particle species of patchy particles such that the patchy particles assemble into a target structure, the method comprising:
receiving a patchy particle model descriptive of a respective patch scheme for at least one particle species of patchy particles, the patchy particle model including a plurality of variables representative of:
a plurality of patch slots defined for each particle species;
a plurality of patch types defined for each particle species;
wherein each patch type of the plurality of patch types is compatible with one other patch type of the plurality of patch types;
wherein each patch slot of the plurality of patch slots is associated with a respective patch type of the plurality of patch types; and
a patch type interaction matrix representative of compatibility between each patch type of the plurality of patch types;
receiving a target lattice structure, the target lattice structure including a plurality of possible lattice positions of a unit cell of the target lattice structure, wherein the unit cell includes one or more patchy particles;
determining a plurality of conditional clauses representative of a plurality of constraints that the one or more variables of the patchy particle model of each of the at least one particle species of patchy particles must satisfy such that the one or more particle species of patchy particles are operable to self-assemble into the target lattice structure based on their respective patch scheme; and
applying the plurality of conditional clauses to the plurality of variables representative of the respective patch scheme to identify one or more solutions to the plurality of variables such that each conditional clause of the plurality of conditional clauses is satisfied.

2. The method of claim 1, further comprising:
simulating the patchy particle model with each solution for each respective particle species to obtain a resultant crystal formation.

3. The method of claim 2, further comprising:
excluding one or more solutions that form a competing structure.

4. The method of claim 1, wherein the plurality of conditional clauses include a first clause, the first clause enforcing a restriction that each patch type of the patchy particle model is compatible with exactly one patch type of the plurality of patch types.

5. The method of claim 1, wherein the plurality of conditional clauses includes a second clause, the second clause enforcing a restriction that each patch slot of the plurality of patch slots is assigned exactly one patch type.

6. The method of claim 1, wherein the plurality of conditional clauses includes a third clause, the third clause enforcing a restriction that each lattice position of the plurality of possible lattice positions is occupied by a single patchy particle species with one assigned orientation.

7. The method of claim 1, wherein the plurality of conditional clauses includes a fourth clause, the fourth clause enforcing a restriction that patch types of patches of the patchy particles that interact within the target lattice structure are operable for binding to one another according to one or more compatibility rules determined by the patch type interaction matrix.

8. The method of claim 1, wherein the plurality of conditional clauses includes a fifth clause, the fifth clause enforcing that each of the plurality of patch slots associated with each lattice position of the unit cell are set to have a patch type of the patch occupying each respective patch slot.

9. The method of claim 1, wherein the plurality of conditional clauses include:
a sixth clause, the sixth clause enforcing that all particle species of the plurality of particle species are included in the target lattice structure; and
a seventh clause, the seventh clause enforcing that all patch types of the plurality of patch types are used in the one or more solutions.

10. The method of claim 1, wherein the one or more solutions is a null solution, the null solution being indicative of an inability of the one or more patchy particle species to self-assemble to a target configuration.

11. A system, comprising:
one or more processors, the one or more processors implementing a module including a plurality of sub-modules, the plurality of sub-modules including:
a first module configured to receive a patchy particle model descriptive of a respective patch scheme for at least one particle species of patchy particles, the patchy particle model including a plurality of variables representative of:
a plurality of patch slots defined for each particle species;
a plurality of patch types defined for each particle species;
wherein each patch type of the plurality of patch types is compatible with one other patch type of the plurality of patch types;
wherein each patch slot of the plurality of patch slots is associated with a respective patch type of the plurality of patch types; and
a patch type interaction matrix representative of compatibility between each patch type of the plurality of patch types;
a second module configured to receive a target lattice structure, the target lattice structure including a plurality of possible lattice positions of a unit cell of the target lattice structure, wherein the unit cell includes one or more patchy particles;
a third module configured to determine a plurality of conditional clauses representative of a plurality of constraints that the one or more variables of the patchy particle model of each of the at least one particle species must satisfy such that the one or more patchy particle species are operable to self-assemble into the target lattice structure based on their respective patch scheme; and
a satisfiability module configured to apply the plurality of conditional clauses to the plurality of variables representative of the patch scheme to identify one or more solutions to the plurality of variables such that each conditional clause of the plurality of conditional clauses is satisfied.

13. The system of claim 11, further comprising a model simulator module, the model simulator module configured to simulate the patchy particle model with each solution for each respective particle species to obtain a resultant crystal formation.

14. The system of claim 13, wherein one or more simulated solutions that form a competing structure are excluded.

15. The system of claim 11, wherein the plurality of conditional clauses include a first clause, the first clause enforcing a restriction that each patch type of the patchy particle model is compatible with exactly one other patch type of the plurality of patch types.

16. The system of claim 11, wherein the plurality of conditional clauses includes a second clause, the second clause enforcing a restriction that each patch slot of the plurality of patch slots is assigned exactly one patch type of the plurality of patch types.

17. The system of claim 11, wherein the plurality of conditional clauses includes a third clause, the third clause enforcing a restriction that each lattice position of the plurality of possible lattice positions is occupied by a single patchy particle species with one assigned orientation.

18. The system of claim 11, wherein the plurality of conditional clauses includes a fourth clause, the fourth clause enforcing a restriction that patch types of patches of the patchy particles that interact within the target lattice structure are operable for binding to one another according to one or more compatibility rules determined by the patch type interaction matrix.

19. The system of claim 11, wherein the plurality of conditional clauses includes a fifth clause, the fifth clause enforcing that each of the plurality of patch slots associated with each lattice position of the unit cell are set to have a patch type of the patch occupying each respective patch slot.

20. The system of claim 11, wherein the plurality of conditional clauses include:
a sixth clause, the sixth clause enforcing that all particle species of the plurality of particle species are included in the target lattice structure; and
a seventh clause, the seventh clause enforcing that all patch types of the plurality of patch types are used in the one or more solutions.
